(19) United States
(12) Patent Application Publication
(10) Pub. No.: US 2006/0159757 A1
(43) Pub. Date: Jul. 20, 2006

(54) Pharmaceutical composition for controlled release of beta-lactam antibiotics in combination with beta-lactamase inhibitors

(75) Inventors: Keith Payne, Accrington (GB); Christian Kiera, Hiddenhausen (DE); Jennifer Dressman, Frankfurt am Main (DE)

Correspondence Address: WELSH & KATZ, LTD, 120 S RIVERSIDE PLAZA, 22ND FLOOR, CHICAGO, IL, 60606, US

(73) Assignee: NextPharma GmbH, Goettingen (DE)

(21) Appl. No.: 11/303,516

(22) Filed: Dec. 16, 2005

(30) Foreign Application Priority Data
Jan. 3, 2005 (DE) DE 102005000719.8

(51) Int. Cl. A61K 31/43; A61K 31/545; A61K 9/26; A61K 31/407

(52) U.S. Cl. 424/470; 514/192; 514/200; 514/210.09

(57) ABSTRACT

The invention relates to a pharmaceutical composition containing a β-lactam antibiotic and/or a pharmaceutically tolerable salt thereof in the form of coated pellets that may optionally contain a β-lactamase inhibitor and/or a pharmaceutically tolerable salt thereof, some or all pellets comprising coatings that dissolve at different pH values depending on the composition of said coatings. This pharmaceutical composition allows the blood plasma level of the β-lactam antibiotic and/or a pharmaceutically tolerable salt thereof to remain above 2 μg/ml for at least 12 hours within a period of 24 hours if administered twice a day.

The invention relates to a pharmaceutical composition for oral administration that—if given twice a day—is capable of releasing a β-lactam antibiotic and/or a pharmaceutically tolerable salt thereof so that the level in the blood plasma, specifically referred to a the minimum inhibition concentration (MIC) does not fall below 2 μg/ml for at least 12 hours within a period of 24 hours.

β-lactam antibiotics, in particular penicillins such as amoxicillin are proven active agents with a broad spectrum of efficacy for treating bacterial infections. It is imperative for efficient treatment that its concentration in the blood plasma does not fall below the minimum inhibition concentration (MIC) for the duration of the treatment.

β-lactam antibiotics are typically administered orally in solid form (tablets or capsules) or as a suspension (powder or tablet for preparing a suspension), and the daily dose is reached by three administrations at regular intervals. This form of administration results in the following development of active agent levels: After each administration, the blood plasma level rises dramatically only to drop relatively fast. Ideally, the next dose should be administered so that the active agent level rises again before it falls below the minimum active agent concentration. This means that the tablets should be taken regularly at intervals that are equally distributed over 24 hours. This has the disadvantage that the patient has to follow a fairly demanding regime resulting in an accordingly high error rate due to late administration, particularly at night, forgetting to take a dose, etc.

This disadvantage is to be overcome by a formulation that only has to be taken twice a day. Treatment duration is considerably reduced as this formulation ensures that the active agent level stays above the MIC. It is also aimed at considerably reducing the risk that bacteria can form a resistance.

Enzymatic degradation of β-lactam antibiotics in the body takes place relatively fast. This degradation can be delayed by adding β-lactamase inhibitors such as clavulanic acid or sulbactam.

Use of polymers suitable for delaying the release of the active agent into the body is another known option. A coating with an enteric polymer is typically used for oral, solid forms such as tablets or granulates. Enteric polymer means that the polymer reacts with water (in the widest sense of the term) at neutral or alkaline pH values, e.g. it may swell or dissolve.

Polymers that dissolve depending on the pH value are typically used as a coating for tablets meant to release their active agent not before reaching the intestinal. EP 1 146 862 describes such a coating with polymers that will not dissolve in gastric or intestinal fluids.

For controlled release, enteric polymers are used that swell at mildly acidic, neutral, or alkaline pH values in aqueous media and form gels. These gels form a diffusion barrier for the active agent. Thus the release rate is eventually controlled by the diffusion though the gel.

Various formulations were specifically developed on this basis for β-lactam antibiotics, especially amoxicillin in combination with clavulanic acid; these release amoxicillin in such a way that the active agent level in the blood plasma remains above the minimum inhibition concentration.

WO 01/00177 describes a tablet formulation with an extended release rate wherein polysaccharides such as cellulose derivatives, xanthan, and carrageenan are used as swellable, gel-forming polymers.

EP 0 776 206, WO 96/04907, EP 0 752 850, U.S. Pat. No. 6,183,778, and EP 0 663 820 describe formulations that contain multiple granulates or layers, these granulates or layers comprising different release rates. Delayed release is achieved by using enteric, swellable polymers.

WO 02/30392 and EP 1 044 680 additionally describe the use of a combination of a salt of amoxicillin and an organic acid that influences the release of the amoxicillin in that it causes further delay.

The systems and methods for delaying release described above cause a temporal delay. But the residence time of substances in the digestive tract is dependent on factors that have a great influence on residence time such as the type and quantity of food taken in. If residence times are very short, a portion of the active agent may be released in a section of the digestive tract where the active agent is no longer or not yet absorbed.

EP 1 146 862 describes the release of the active agent at a specific place in the digestive tract. It is preferably released in the lower ileum and in the colon. This is achieved by pressing very small particles that are coated with a polymer not soluble by gastric and intestinal fluids into compact tablets with a specific porosity. This makes the tablet decompose slower. Eventually, the release rate depends on time here as well.

WO 02/060415 describes a multiparticulate form of medication for treating Crohn's disease or chronic ulcerative colitis that is suited for release of the active agent at specific places in the digestive tract. Various pellets are described that are coated with multiple layers of swelling and dissolving polymers that cause an even release of the active agent in the small intestine and colon only. The disadvantage of this concept is that it does not allow release in the stomach.

Unlike WO 02/060415 that includes a formulation with active agent release at the acute source of the infection, the target of using β-lactam antibiotics is to achieve a systemic effect through suitably high levels of it in the blood plasma for a specific period of time.

To reliably avoid falling below the minimum inhibition concentration of the β-lactam antibiotic, the active agent should not depend on residence time in the digestive tract but directly on its location in the digestive tract. In this way, the entire digestive tract that is capable of absorbing the β-lactam antibiotic—i.e. the stomach and the respective portions in the intestines—should be used for absorption.

It is therefore the object of the invention to provide a pharmaceutical composition in the form of coated pellets for oral administration with a β-lactam antibiotic, optionally in combination with a β-lactamase inhibitor, wherein the release of the active agent depends on the location in the digestive tract.

This object is achieved by a pharmaceutical composition in the form of coated pellets where each pellet contains a β-lactam-antibiotic and/or a pharmaceutically tolerable salt thereof, and optionally an additional β-lactamase inhibitor and/or a pharmaceutically tolerable salt thereof, and where some or all pellets comprise coatings that dissolve at various pH values depending on the composition of the coating(s). This pharmaceutical composition allows the blood plasma level of the β-lactam antibiotic and/or a pharmaceutically tolerable salt thereof to remain above 2 μg/ml for at least 12 hours within a period of 24 hours if administered twice a day.

The pharmaceutical composition preferably includes at least three pellet types A, B, and C with different compositions in one dosing unit. The dosing units in the pharmaceutical composition according to the invention preferably are sachets, tablets, or capsules that are administered twice daily.

The digestive tract has different pH values depending on the respective section. For example, the pH value in the upper section of the stomach is between 1 and 3 while in the lower section it is between 3 and 6. The pH value in the duodenum is approx. 5, in the small intestine approx. 6.5, and in the colon approx. 7.5. It is thus possible to use coatings that dissolve depending on the pH value to achieve controlled release of the active agent at a specific location. Uniform release across the entire section of the digestive tract that is capable of absorption can be achieved by combining various pellets that are coated so that their coating dissolves at different pH values. The β-lactam antibiotic preferably is a penicillin, more preferably an aminopenicillin, most preferably amoxicillin or ampicillin. The β-lactamase inhibitor preferably is clavulanic acid or a pharmaceutically tolerable salt thereof, most preferably potassium clavulanate.

In a preferred embodiment of the invention, pellet type A contains 25 to 60 wt %, preferably 30 to 45 wt %, most preferably 40 wt % of the overall β-lactam antibiotic content.

Pellet type B contains 1 to 70 wt %, preferably 10 to 35 wt %, most preferably 20 wt % of the overall β-lactam antibiotic content.

Furthermore, pellet type C contains 1 to 70 wt %, preferably 30 to 45 wt %, most preferably 40 wt % of the overall β-lactam antibiotic content.

In a preferred embodiment, the β-lactamase inhibitor and/or a pharmaceutically tolerable salt thereof is at least contained in pellet type A. It is most preferred that the inhibitor is exclusively contained in the type A pellets.

In a preferred embodiment of the invention, the pellet cores are from 0.8 to 1.8 mm in diameter.

The type A pellets have one or several coatings that dissolve in gastric juice or in water. Preferably, the coating is to release the active agent in the stomach so that an initial steep rise in the blood plasma concentration is achieved after administration. The coatings consist of polymers selected from the group consisting of modified celluloses and derivatives thereof, polyvinyl pyrrolidones as well as polyacrylic and polymethacrylic acids and polyvinyl acetates and derivatives thereof that dissolve in gastric juice or in water. In a preferred embodiment, the coatings are selected from the group consisting of polyacrylic and polymethacrylic acids and derivatives thereof. The most preferred coating is poly(butylmethacrylate-co-(2-dimethylaminoethyl)methacrylate-co-methyl methacrylate) that has a molar ratio (MR) among the individual polymer components of 1:2:1, at a molecular weight (MW) of 150,000 g/mol [Eudragit E].

In another preferred embodiment, the coatings of the type A pellets are selected from the group consisting of hydroxypropylmethyl cellulose [HPMC] and hydroxypropyl cellulose [HPC].

The factors that determine the dissolution rate of the coating include the thickness of the layer. Thicknesses of polymer layers are between 5 and 80 μm to achieve the desired release rates of the active agents.

The type B pellets have one or several coatings that dissolve at pH values above pH 5, preferably between 5.5 and 7, most preferably between 5.8 and 6.5. This coating allows controlled active agent release in the section between the jejunum and ileum. Accordingly, levels in the blood plasma rise again over time as the released active agent is absorbed. The coatings consist of polymers selected from the group consisting of modified celluloses and derivatives thereof, polyvinyl pyrrolidones as well as polyacrylic and polymethacrylic acids and polyvinyl acetates and derivatives thereof. In a preferred embodiment, the coatings are selected from the group consisting of polyacrylic and polymethacrylic acids and derivatives thereof. The most preferred coatings are selected from the group of poly(methacrylic acid-co-methyl methacrylate) with a molar ratio (MR) among the individual polymer components of 1:1 at a molecular weight (MW) of 135,000 g/mol and poly(methacrylic acid-co-ethylacrylate) (MR=1:1, MW 250,000 g/mol) [Eudragit L].

The preferred layer thicknesses are in the range from 5 to 80 μm, preferably between 30 and 50 μm.

The type C pellets have one or several coatings that dissolve at pH values above pH 6.5, preferably between 6.5 and 7.5, most preferably between 6.7 and 7.3. This coating allows controlled active agent release near the colon. The level in the blood plasma rises again accordingly as the released active agent is absorbed. The coatings consist of polymers selected from the group consisting of modified celluloses and derivatives thereof, polyvinyl pyrrolidones as well as polyacrylic and polymethacrylic acids and polyvinyl acetates and derivatives thereof. In a preferred embodiment, the coatings are selected from the group consisting of polyacrylic and polymethacrylic acids and derivatives thereof. The most preferred coatings are selected from the group consisting of poly(methacrylic acid-co-methyl methacrylate) (MR 1:2, MW 135,000 g/mol) [Eudragit S], poly(methacrylic acid-comethyl methacrylate) (MR 1:1, MW 135,000 g/mol) and poly(methacrylic acid-co-ethylacrylate) (MR 1:1, MW 250,000 g/mol) [Eudragit L] and/or a combination thereof.

The preferred layer thicknesses are in the range from 5 to 80 μm, preferably between 30 and 50 μm.

The composition according to the invention may further include a pellet type D having one or several coatings that dissolve at pH values above pH 7.0, preferably between 7.0 and 8.0, most preferably between 7.3 and 7.8. This coating allows controlled active agent release near the colon. Thus another rise in active agent levels by absorption can be achieved. The coatings consist of polymers selected from the group consisting of modified celluloses and derivatives thereof, polyvinyl pyrrolidones as well as polyacrylic and polymethacrylic acids and polyvinyl acetates and derivatives thereof. In a preferred embodiment, the coatings are selected from the group consisting of polyacrylic and polymethacrylic acids and derivatives thereof. The most preferred coatings are selected from the group of poly(methylacrylate-co-methyl methacrylate-co-methacrylic acid)(7:3:1, MW 220,000 g/mol) [Eudragit FS].

The preferred layer thicknesses are in the range from 5 to 80 μm, preferably between 30 and 50 μm.

As release is controlled, the composition of the pellet type may be varied depending on the quantity of active agent required for maintaining or increasing the active agent level in the plasma.

To slow down the enzymatic degradation of the β-lactam antibiotic, one of the pellet types, preferably type A, may contain the β-lactamase inhibitor, preferably clavulanic acid or a pharmaceutically tolerable salt thereof, most preferably potassium clavulanate. The total quantity in the affected type is at a molar β-lactam antibiotic to β-lactamase inhibitor ratio from 2:1 to 18:1, preferably from 2:1 to 4:1.

Other embodiments may include the common adjuvants for making pellets and coating particles. These adjuvants can be located in the pellet core or in the coating agent (coating). Agents used can be organic or aqueous solvents, suspending or dispersing agents or mixtures thereof. Isopropanol, acetone and water are among the preferred agents.

In a preferred embodiment of the invention, the core of the type A pellets contains amoxicillin trihydrate and potassium clavulanate triturated with (microcrystalline) cellulose or silicon dioxide, klucel (hydroxypropylcellulose) and isopropanol. The isopropanol is removed by drying during preparation. In a preferred embodiment, magnesium stearate and isopropanol are used in addition to poly(butylmethacrylate-co-(2-dimethylaminoethyl)methacrylate-co-methyl methacrylate) [Eudragit E] for coating the type A pellets, the isopropanol being removed later in the manufacturing process.

In a preferred embodiment of the invention, the cores of pellet types B, C, D contain (microcrystalline) cellulose and water in addition to amoxicillin (trihydrate), the water being later removed by drying.

In a preferred embodiment, the coating of the type B pellets contains triethyl citrate, magnesium stearate, isopropanol and water in addition to Eudragit L. The latter two substances are removed later in the process.

In a preferred embodiment, the coating of the type C pellets contains softeners and separating agents as well as isopropanol and water in addition to Eudragit S or Eudragit S and L. The latter two substances are removed later in the process.

In a preferred embodiment, the coating of the type D pellets contains softeners as well as isopropanol and water in addition to Eudragit FS. The latter is removed later in the process.

Common coating methods can be used for coating the pellets.

In a preferred embodiment of the invention, the coatings (films) are applied to the pellets using a fluidized-bed reactor. The respective polymer or polymer mixture is dissolved using organic solvents to become a clear solution, then optional adjuvants may be added. The resulting solution or suspension is then atomized.

If film-forming agents are processed in aqueous media, the film-forming agent, such as Eudragit FS, is present in an aqueous dispersion. Adjuvants may optionally be added. Then the dispersion is atomized.

Atomized solutions preferably have a solids content of 5 to 25% depending on the resulting stickiness.

The mixture of the various pellets (granulate) can be dosed in hard gelatin capsules. Other embodiments include granulates formulated in sachets for preparing a drinkable solution or a suspension. The granulate according to the invention may therefore contain the adjuvants that are common in these forms, for example, suspension stabilizers. Embodiments may additionally include flow improvers such as talc, magnesium stearate, and silicon dioxide, thickeners, sweeteners, as well as flavor-enhancing agents.

The invention will be explained with reference to examples below without limiting the invention to said examples.

### EXAMPLES

The pellets are prepared using common methods by mixing the components, extrusion, and pelleting. Subsequently, they are coated with the polymers using one of the common methods.

### Example 1

Granulate with Pellet Types A, B, and C

The type A pellets contain amoxicillin in quantities as indicated in Table 1 and up to 125 mg of potassium clavulanate. The coating consists of poly(butylmethacrylate-co-(2-dimethylaminoethyl)methacrylate-co-methyl methacrylate) at a molar ratio (MR) among the monomeric components of 1:2:1, the polymer having a molecular weight (MW) of 150,000 g/mol [Eudragit E]. The pellets are coated with the polymers using one of the common methods.

The type B pellets contain amoxicillin quantities as indicated in Table 1 and are coated with a combination of poly(methacrylic acid-co-methyl methacrylate) (MR 1:1, MW 135,000 g/mol) and poly(methacrylic acid-co-ethyl acrylate) (MR 1:1, MW 250,000 g/mol) [Eudragit L]. The pellets are coated with the polymers using one of the common methods.

The type C pellets contain amoxicillin in quantities as indicated in Table 1. They are either coated with poly(methacrylic acid-co-methyl methacrylate) (MR 1:2, MW 135,000 g/mol) [Eudragit S] only or with a combination of poly(methacrylic acid-co-methyl methacrylate) (MR 1:1, MW 135,000 g/mol) and poly(methacrylic acid-co-ethyl acrylate) (MR 1:1, MW 250,000 g/mol) [Eudragit L]. The pellets are coated with the polymers using one of the common methods.

Table 1 lists various granulate compositions in which the pellets of type A, B, and C contain different quantities of amoxicillin. The weight is given for the overall quantity per dosing unit.

TABLE 1

| | | | | Overall | | | |
|---|---|---|---|---|---|---|---|
| | | | | quantity per | | | |
| | | | | dosing unit | | | |
| Pellet type | A⁽⁺⁾ | B | C | [mg] | A [%] | B [%] | C [%] |
| Amoxicillin | 600 | 300 | 600 | 1,500 | 40.0 | 20.0 | 40.0 |
| [mg] | 700 | 300 | 500 | 1,500 | 47.0 | 20.0 | 33.0 |
| (trihydrate) | 500 | 300 | 700 | 1,500 | 33.0 | 20.0 | 47.0 |
| | 700 | 200 | 600 | 1,500 | 47.0 | 13.0 | 40.0 |
| | 600 | 400 | 500 | 1,500 | 40.0 | 27.0 | 33.0 |
| | 700 | 400 | 700 | 1,800 | 39.0 | 22.0 | 39.0 |
| | 800 | 200 | 800 | 1,800 | 44.5 | 11.0 | 44.5 |
| Coating* | Eudragit E | Eudragit L | Eudragit S, or a | | | | |
| | | | combination of | | | | |
| | | | Eudragit S and L | | | | |

*Eudragit coatings manufactured by: Degussa
⁽⁺⁾The type A pellets contain 125 mg of potassium clavulanate

### Example 2

Granulate with Pellet Type A and Another Pellet Type

The pellet types A, B, or C are prepared like in Example 1 and contain amoxicillin in quantities as indicated in Table 2. The pellets of type D that contain amoxicillin in quantities as indicated in Table 2 are coated with poly(methylacrylate-co-methyl methacrylate-co-methacrylic acid) (MR 7:3:1, MW 220,000 g/mol) [Eudragit FS]. The pellets are coated with the polymers using one of the common methods.

Table 2 lists various granulate compositions in which the pellets contained different amoxicillin quantities. The weight is given for the overall quantity per dosing unit.

TABLE 2

| | | | Total | | B or C |
|---|---|---|---|---|---|
| | | B or | quantity | | or D⁽⁺⁺⁾ |
| Pellet type | A⁽⁺⁾ | C or D⁽⁺⁺⁾ | [mg] | A [%] | [%] |
| Amoxicillin | 750 | 750 | 1,500 | 50.0 | 50.0 |
| [mg] | 800 | 700 | 1,500 | 53.0 | 47.0 |
| (trihydrate) | 700 | 800 | 1,500 | 47.0 | 53.0 |
| | 600 | 900 | 1,500 | 40.0 | 60.0 |
| | 500 | 1000 | 1,500 | 33.0 | 67.0 |

⁽⁺⁾The type A pellets contain 125 mg of potassium clavulanate
⁽⁺⁺⁾The type D pellets are coated with Eudragit FS.

### Example 3

The pellet types A, B, and C are mixed with ingredients such as sweeteners, flavors and dyes as well as suspending agents and packed in sachets.

### Example 4

Blood Plasma Level of Amoxicillin from a Granulate According to the Invention

The concentration of amoxicillin in the blood plasma over time was simulated based on the release profiles of pellet types A, B and C obtained from dissolution tests in aqueous solution at the respective pH values.

FIG. 1: Blood plasma level of amoxicillin after the intake of a preparation according to the invention.

[image]

1. A pharmaceutical composition in the form of coated pellets wherein each pellet contains a β-lactam antibiotic and/or a pharmaceutically acceptable salt thereof and, optionally, an additional β-lactamase inhibitor and/or a pharmaceutically acceptable salt thereof, and wherein one or more pellets comprise coatings that dissolve at various pH values depending on the composition of the respective coating(s) so that the level of the β-lactam antibiotic and/or a pharmaceutically acceptable salt thereof in the blood plasma remains above 2 μg/ml for at least 12 hours within a period of 24 hours.

2. The pharmaceutical composition according to claim 1 wherein the composition includes at least three differently composed pellet types A, B, and C in one dosage unit.

3. The pharmaceutical composition according to claim 2 wherein the type A pellets comprise 25 to 60 percent by weight of the overall β-lactam antibiotic content.

4. The pharmaceutical composition according to claim 3 wherein the type A pellets comprise 30 to 45 percent by weight of the overall β-lactam antibiotic content.

5. The pharmaceutical composition according to claim 2 wherein the type B pellets comprise 1 to 70 percent by weight of the overall β-lactam antibiotic content.

6. The pharmaceutical composition according to claim 5 wherein the type B pellets comprise 10 to 35 percent by weight of the overall β-lactam antibiotic content.

7. The pharmaceutical composition according to claim 2 wherein the type C pellets comprise 1 to 70 percent by weight of the overall β-lactam antibiotic content.

8. The pharmaceutical composition according to claim 7 wherein the type C pellets comprise 30 to 45 percent by weight of the overall β-lactam antibiotic content.

9. The pharmaceutical composition according to claim 2 wherein the type A pellets comprise 30 to 45 percent by weight, the type B pellets comprise 10 to 35 percent by weight and the type C pellets comprise 30 to 45 percent by weight of the overall β-lactam antibiotic content.

10. The pharmaceutical composition according to claim 1 wherein the β-lactam antibiotic is a penicillin.

11. The pharmaceutical composition according to claim 10 wherein the penicillin is an aminopenicillin.

12. The pharmaceutical composition according to claim 11 wherein the aminopenicillin is ampicillin or amoxicillin.

13. The pharmaceutical composition according to claim 2 wherein the β-lactamase inhibitor is at least contained in pellet type A.

14. The pharmaceutical composition according to claim 13 wherein the β-lactamase inhibitor is clavulanic acid or a pharmaceutically acceptable salt thereof.

15. The pharmaceutical composition according to claim 1 wherein the β-lactam antibiotic is amoxicillin and the β-lactamase inhibitor is clavulanic acid or a pharmaceutically acceptable salt thereof.

16. The pharmaceutical composition according to claim 2 wherein the type A pellets have one or several coatings that dissolve in gastric juice and/or water.

17. The pharmaceutical composition according to claim 16 wherein the coatings of the type A pellets are selected from the group consisting of cellulose derivatives, polyvinylpyrrolidones as well as polyacrylic and polymethacrylic acids, polyvinylacetates and derivatives and combinations thereof that dissolve in gastric juice and/or in water.

18. The pharmaceutical composition according to claim 17 wherein the coatings of the type A pellets are selected from the group consisting of polyacrylic and polymethacrylic acids and derivatives and combinations thereof.

19. The pharmaceutical composition according to claim 18 wherein the coating of the type A pellets is poly(butylmethacrylate-co-(2-dimethyl aminoethyl) methacrylate-co-methylmethacrylate) (1:2:1, MW 150,000 g/mol).

20. The pharmaceutical composition according to claim 17 wherein the coatings of the type A pellets are selected from the group consisting of hydroxypropylmethylcellulose and hydroxypropylcellulose.

21. The pharmaceutical composition according to claim 2 wherein the type B pellets have one or several coatings that dissolve at pH values above pH 5.

22. The pharmaceutical composition according to claim 21 wherein the type B pellets have one or several coatings that dissolve at pH values between 5.5 and 7.0.

23. The pharmaceutical composition according to claim 22 wherein the coatings of the type B pellets dissolve at pH values between 5.8 and 6.5.

24. The pharmaceutical composition according to claim 21 wherein the coatings of the type B pellets are selected from the group consisting of cellulose derivatives, polyvinylpyrrolidones as well as polyacrylic and polymethacrylic acids and polyvinylacetates and derivatives and combinations thereof.

25. The pharmaceutical composition according to claim 24 wherein the coatings of the type B pellets are selected from the group consisting of polyacrylic and polymethacrylic acids and derivatives and combinations thereof.

26. The pharmaceutical composition according to claim 25 wherein the coatings of the type B pellets are selected from the group consisting of poly(methacrylicacid-co-methylmethacrylate) (1:1, MW 135,000 g/mol) and poly(methacrylic acid-co-ethylacrylate) (1:1, MW 250,000 g/mol).

27. The pharmaceutical composition according to claim 2 wherein the type C pellets have one or several coatings that dissolve at pH values above pH 6.5.

28. The pharmaceutical composition according to claim 27 wherein the type C pellets have one or several coatings that dissolve at pH values between 6.5 and 7.5.

29. The pharmaceutical composition according to claim 28 wherein the coatings of the type C pellets dissolve at pH values between 6.7 and 7.3.

30. The pharmaceutical composition according to claim 27 wherein the coatings of the type C pellets are selected from the group consisting of cellulose derivatives, polyvinylpyrrolidones as well as polyacrylic and polymethacrylic acids and polyvinylacetates and derivatives and combinations thereof.

31. The pharmaceutical composition according to claim 30 wherein the coatings of the type C pellets are selected from the group consisting of polyacrylic and polymethacrylic acids and derivatives and combinations thereof.

32. The pharmaceutical composition according to claim 31 wherein the coatings of the type C pellets are selected from the group consisting of poly(methacrylicacid-co-methylmethacrylate) (1:2, MW 135,000 g/mol), poly(methacrylicacid-co-methylmethacrylate) (1:1, MW 135,000 g/mol) and poly(methacrylicacid-co-ethylacrylate) (1:1, MW 250,000 g/mol) and/or a combination thereof.

33. The pharmaceutical composition according to claim 2, characterized in that it contains another pellet type D.

34. The pharmaceutical composition according to claim 33 wherein the type D pellets have one or several coatings that dissolve at pH values above pH 7.0.

35. The pharmaceutical composition according to claim 34 wherein the coatings of the type D pellets dissolve at pH values between 7.0 and 8.0.

36. The pharmaceutical composition according to claim 35 wherein the coatings of the type D pellets dissolve at pH values between 7.3 and 7.8.

37. The pharmaceutical composition according to claim 34 wherein the coatings of the type D pellets are selected from the group consisting of cellulose derivatives, polyvinylpyrrolidones as well as polyacrylic and polymethacrylic acids and polyvinylacetates and derivatives and combinations thereof.

38. The pharmaceutical composition according to claim 37 wherein the coatings of the type D pellets are selected from the group consisting of polyacrylic and polymethacrylic acids and derivatives and combinations thereof.

39. The pharmaceutical composition according to claim 38 wherein the coatings of the type D pellets are selected from the group consisting of poly(methacrylate-co-methylmethacrylate-co-meth-acrylicacid) (7:3:1, MW 220,000 g/mol).
